(19) United States
(12) Patent Application Publication
(10) Pub. No.: US 2015/0269229 A1
(43) Pub. Date: Sep. 24, 2015

(54) Virtual Pointer and Annotations Through Real Time Communications

(71) Applicant: NUREP, INC., San Jose, CA (US)

(72) Inventors: Sainath Shenoy, San Jose, CA (US); Paul Schultz, San Jose, CA (US); Adam John, San Jose, CA (US)

(73) Assignee: NUREP, INC., San Jose, CA (US)

(21) Appl. No.: 14/662,189

(22) Filed: Mar. 18, 2015

Related U.S. Application Data
(60) Provisional application No. 61/954,808, filed on Mar. 18, 2014.

(51) Int. Cl. G06F 17/30; A61B 19/00; H04L 29/08; H04L 29/06; G06F 3/0354; G06F 17/24

(52) U.S. Cl. CPC G06F 17/30525; G06F 3/03545; G06F 17/241; H04L 67/02; H04L 65/60; A61B 19/5244; A61B 2019/5268

(57) ABSTRACT

An apparatus, system, and method, is described for the capturing and processing of virtual annotations that identify features on medical devices; the annotations may be captured using mobile computing devices, such as smartphones, and displayed on mobile display devices, such as, tablets; and communicated across a network using standard internet protocols.

### CROSS REFERENCE TO RELATED APPLICATIONS

The present application claims the benefit of U.S. Provisional Patent Application No. 61/954,808, filed on Mar. 18, 2014 and entitled “Virtual pointer and annotations through real time communications” which is herein incorporated by reference in its entirety.

### FIELD

The present inventive subject matter provides for methods and the supporting the virtual annotation of surgical instrument systems in particular, and the generalized virtual annotation of instruments in particular.

### BACKGROUND

Real time virtual pointer and annotation technology has practical uses in many professional endeavors.

For example there are certain fields that involve use of delicate instruments that are operated by skilled professionals, such as medical surgery. In the case of surgery, remote monitoring is used as an important training tool for residents. Also, remote monitoring can also be used by medical device suppliers as they assist doctors while performing surgeries.

For surgeries, it is not uncommon to have numerous instruments laid out on the medical instrument table. While the surgeon intuitively knows which instrument is in use, it is sometimes difficult for the remote viewer to identify and keep track of similar instruments. This problem is more apparent when the surgical monitoring device changes position and the field view of the surgical table, a common problem with mobile devices and their inherent portability. Also in the medical field compliance with privacy laws is very important. Therefore it is of prime necessity that any computer equipment have secure internet connections and restricted access to any stored data.

WebRTC is a technology for performing real-time video and audio communication in a browser, which can implement an audio/video call function between browsers or between a browser and a conventional communications terminal. For example, a video conference can be performed by using two different browsers supporting the WebRTC function. WebRTC technical specifications are specified by the Internet Engineering Task Force (IETF) and the World Wide Web consortium (W3C) together. JSON is an acronym for JavaScript Object Notation, which is an open standard for transmitting attribute value pairs.

Electronic video data often includes interactive features related to images appearing in video output. For example, Adobe™ Flash™, Blu-ray™, and other media player applications support layered video, overlays, and similar features that can be included with video data. Using such features, video data may incorporate objects within the picture frame itself that are responsive to user input to link to further information. For example, video data may be configured with areas or objects that a user may select using input from a touchscreen, pointing device, or the like. In response to detecting user selection input directed to a preconfigured object appearing in a video, a computer may take some predetermined action based on the identity of the selected object. For example, the computer may obtain and display some additional information about the interactive object in a separate window or display area.

Embedded interactivity, however, has not become commonplace even in environments that readily support user interactivity, for example, in personal computers, notepad computers, smartphones, and so forth. Prior methods for preparing interactive content embedded in video content may require a significant amount of manual configuration and planning. Such requirements may discourage the creation of interactive content and limit its distribution. In addition, much video content is still primarily viewed on non-interactive platforms such as televisions, which may further reduce the incentives for producing interactive video content.

### SUMMARY

The present inventive subject matter provides for methods and the supporting systems that provide for sending and receiving data over a real time communication channel.

In one embodiment, the system includes a first communication device equipped with a display and camera, coupled to a communication network, and a second communication device equipped with display and camera, coupled to a communication network.

In another embodiment, the first device can send specific data, which may be in the form of x and y coordinates of the display which may be in the form of JSON, to a data server, in which the second communication device listens to changes to the x and y coordinates and receives those changes from the server, displaying them accordingly.

In another embodiment, the inventive subject matter includes methods, systems and programs for sending and receiving data over a real time communication channel. The system includes a first communication device equipped with a display and camera, coupled to a communication network, and a second communication device equipped with display and camera, coupled to a communication network. In the same or another embodiment, the first device can send specific data, which may be in the form of x and y coordinates of the display which may be in the form of JSON, in which the second communication device listens to changes to the x and y coordinates and receives those changes and displays them accordingly.

Also described is an apparatus for the virtual annotation of surgical instruments with an image frame grabber, the image frame grabber capable of receiving and processing a multiplicity of sequential images captured by an image sender and a transmitting display device, the transmitting display device electronically that is connected to the image frame grabber capable of receiving images from the image sender; and an image annotator, with the image annotator capable of tagging a multiplicity of target points on the images transmitted by the image frame grabber; a reference point coordinator, the reference point coordinator capable of storing a multiplicity of reference points that are correlated to the multiplicity of instruments; and a reference point overlayer, that the reference point over layer operably coupled to a receiving display device; such that the sequential images of the image sender are visually presented on the receiving display with the reference points overlaid on the receiving display device.

Also described is a system for the storage of virtual annotations that identify features on surgical instruments having a means for capturing and electronically storing in a cloud-based database a series of user annotated surgical instrument feature overlays captured from a sender; and also with a means for retrieving from the cloud-based database the series of user annotated surgical instrument features and displaying on a receiver captured from the sender; so that the images that are displayed on the receiver are similar to those that are displayed on the sender with the user annotated surgical instrument feature overlays.

Also described is a method for the virtual annotation of surgical instruments that involves the capturing, storing, and displaying a series of image frames on a first mobile display device by an integrated camera; receiving user annotations on the first mobile display device of the image frames that identify and correlate features on the image frames, reprojecting and overlaying those annotations on the first mobile display device; and storing on a networkable server the series of image frames and the annotations; and transmitting the series of image frames and annotations to a second mobile display device; and displaying the series of image frames and annotations on a second mobile display device; so that a viewer of the second mobile display device observes in near real time the series of image frames that were captured by the integrated camera.

The above mentioned inventive subject matter is applicable in various medical specialities like Cardiaology, Orthopedic, Spinal, Neurology, etc. and also for implantable medical devices, medical equipment, diagnostics used in X-rays or ultrasounds or angiograms. The virtual annotation technology is mainly used for visual guidance throughout the operating room, catheter lab or any other patient care area where a surgery/procedure may occur. Ideally virtual annotations are used for the purpose of providing guidance during a medical device related procedure in any patient scenario.

The above-mentioned and additional features of the present invention are further illustrated in the detailed description. All references disclosed herein, including U.S. patents and patent applications, are hereby incorporated by reference in their entirety.

### BRIEF DESCRIPTION OF THE DRAWINGS

FIG. 1 is a system diagram of an embodiment of the surgical instrument annotation system.

FIG. 2 is a diagram of the data interactions between the sending display and the receiving display.

FIG. 3 is a flow diagram of the describing the data interactions between the sending display and the receiving display.

FIG. 4 is a screenshot of one embodiment in a particular use case of the present invention.

FIG. 5 is a screenshot of an embodiment showing the capturing of snapshots and annotations.

### DETAILED DESCRIPTION

The following will describe in detail several preferred embodiments of the present invention. These embodiments are provided by way of explanation only, and thus, should not unduly restrict the scope of the invention. In fact, those of ordinary skill in the art will appreciate upon reading the present specification and in conjunction with the present drawings that other there are equivalent variations and modifications, and that numerous variations of these embodiments may be employed, used and made without departing from the scope and spirit of the disclosed subject matter.

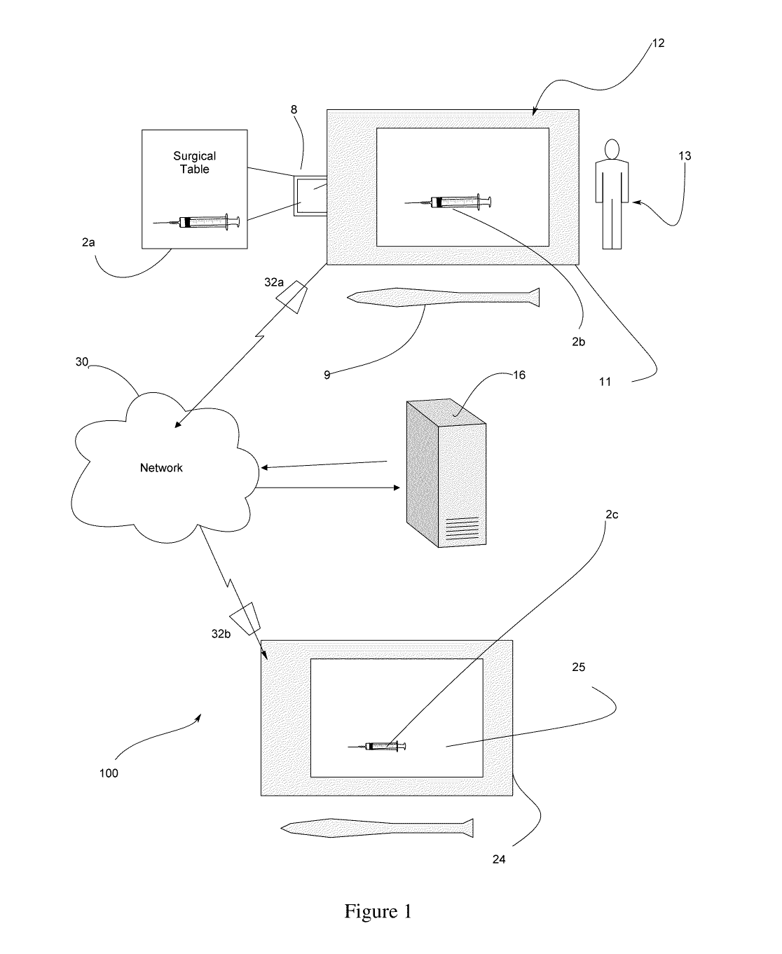

Now referring to FIG. 1 which provides a system diagram 100 of an embodiment of the surgical annotation system. The user 13 may be on a mobile device 12 with a camera 8 that is connected to a communication network link 32a. The sender 12 with a camera 8 is connected to a receiver 24 connected through a communication network link 32b. An image capture of the surgical instrument 2a with the camera 8, is transmitted to the sender screen 11 and shown as a reproduced surgical instrument 2b, and the receiver screen 25 and shown as reproduced surgical instrument 2c. The sender stylus 9 may be used annotate the reproduced surgical instrument 2b with an identifiable electronic marker. The identifiable electronic marker annotation is then reproduced on the surgical instrument 2c on the receiver screen 25.

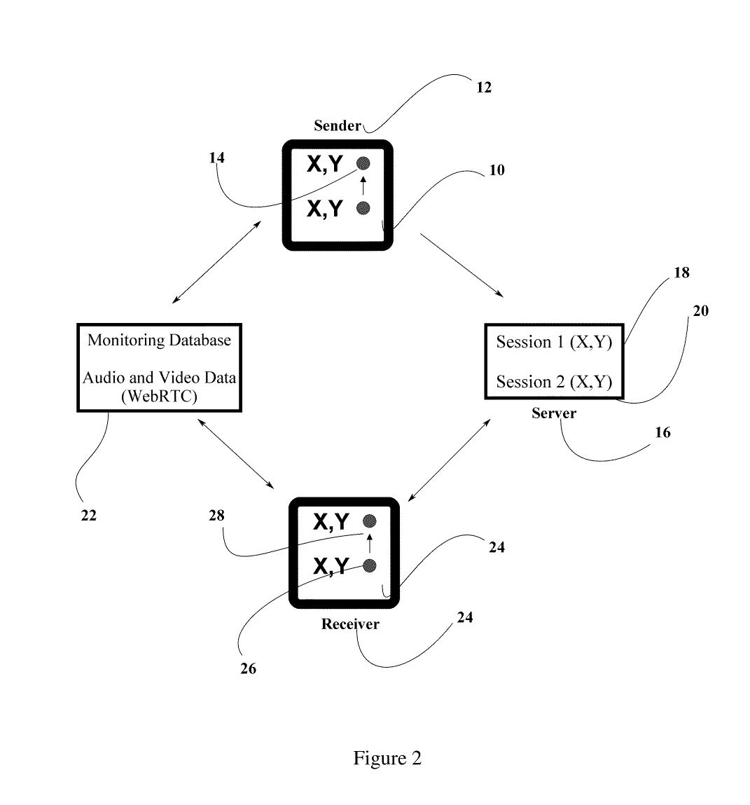

Now referring to FIG. 2 which illustrates the data interaction between the sender 12 and receiver 24. The sender 12 and receiver 24 may be connected to each sharing video, audio and data through web real time communication (WebRTC) application programming interface (API) which may be encrypted through AES 128 bit.

While sender and receiver are interfacing and monitoring 22 during a WebRTC communication on their mobile devices, the sender may have the ability to touch the mobile device screen 10. When the user 13 touches the screen, a visual may be displayed, such as a dot or circle 14. This interaction may update the X, Y JSON coordinates 18 in a server 16. The changes to the X, Y JSON coordinates 18 may cause the server to update the coordinates for receiver X, Y JSON coordinates 20. The receiver 24 could be listening for changes to the X, Y JSON coordinates 20. When changes occur to X, Y JSON coordinates 20, a visual display occurs 26. This visual display could be in the form of a dot or circle. The visual display could disappear or stay visible for a specified period of time. The visual display could move when the sender 12 touches the screen in another location with different X, Y coordinates 14. This causes the server 16 to change X, Y JSON coordinates 18, 20 which in turn changes the visible display on the receiver to 28. The visual display could be dragged to represent a visual annotation. The visual display could also be removed by the user through another interaction. The visual display could be modified to show any diameter, length, width or shape.

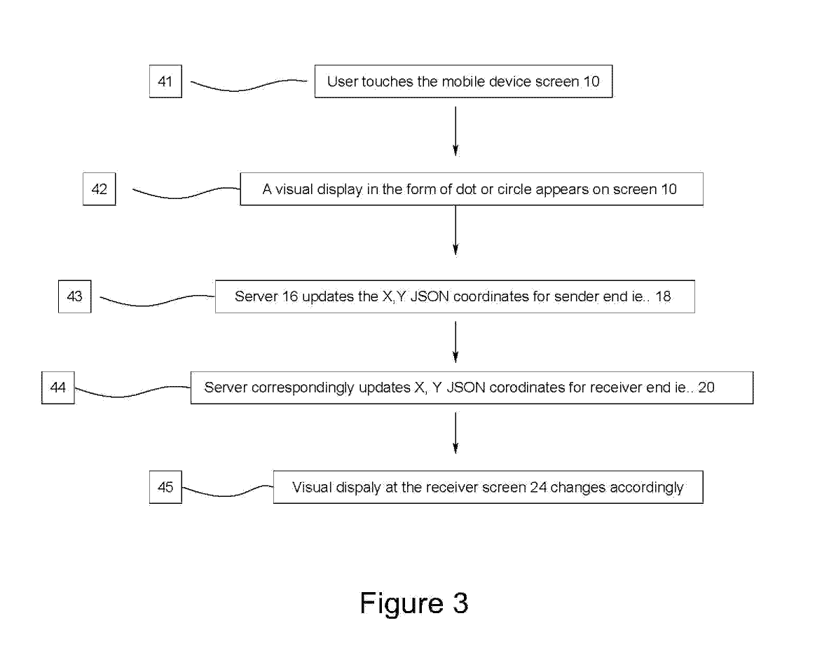

Now referring to FIG. 2 and FIG. 3, a flowchart of the data interaction between the sender 12 and receiver 24 is depicted in FIG. 3. As the user touches the mobile device screen 10 as in 41 a visual display in the form of dot or circle appears on screen 10 as shown in 42. In response to these changes the server 16 updates the X, Y JSON coordinates for sender ie. 18 as shown in 43. Immediately the server updates the X, Y JSON coordinates for receiver end ie. 20 as shown in 44. Accordingly the visual display for receiver screen 24 changes as shown in 45.

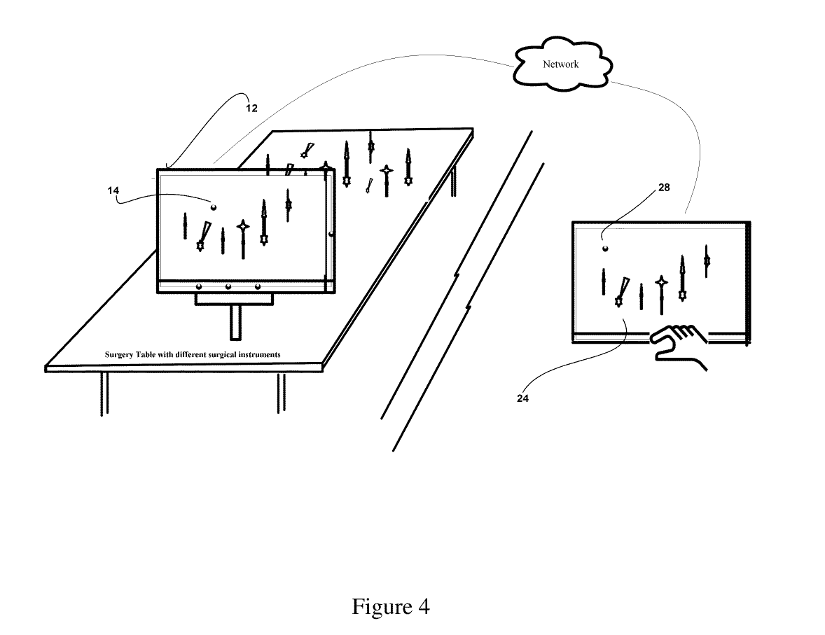

FIG. 4 illustrates one implementation of the invention as described in FIG. 2. Note the X, Y JSON coordinates represent a visual display, such as a dot or circle, on the sender 12 and receiver 24 mobile devices.

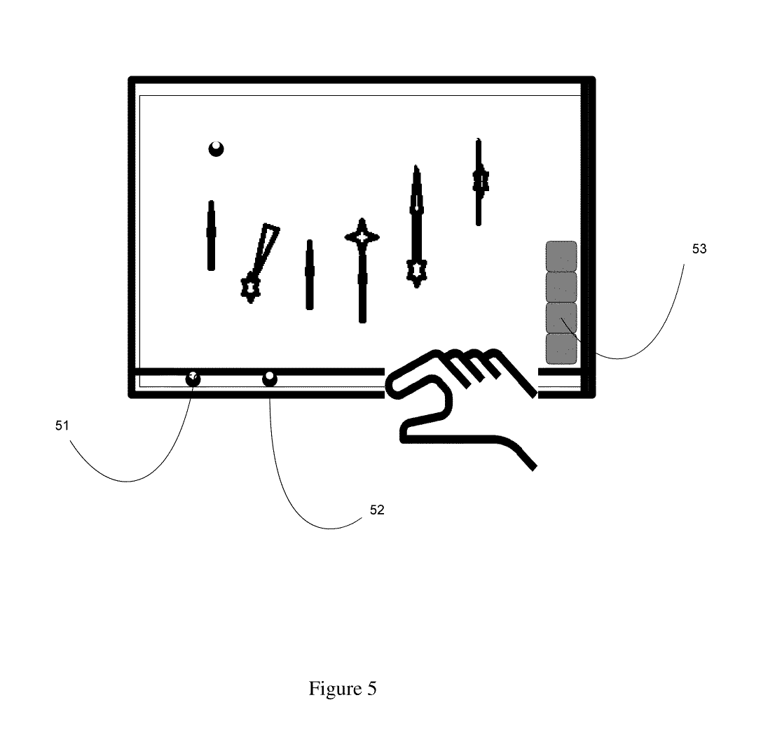

The annotations done during a session are also encrypted. As shown in FIG. 5 the user has an option to take a snapshot by pressing button 51 of the secure live video and take annotations over the still image by pressing button 52. These images can be saved and if there are multiple images then it can be saved onto an image reel as shown in 53. Although the images can be saved on to an image sequence or image reel, at the end of the session, the images and the annotations are deleted

The many aspects and benefits of the invention are apparent from the detailed description, and thus, it is intended for the following claims to cover all such aspects and benefits of the invention which fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

I claim:

1. An apparatus for the virtual annotation of medical devices comprising:
an image frame grabber, the image frame grabber capable of receiving and processing a multiplicity of sequential images captured by an image sender;
a transmitting display device, the transmitting display device electronically connected to the image frame grabber capable of receiving images from the image sender;
an image annotator, the image annotator capable of tagging a multiplicity of target points on the images transmitted by the image frame grabber;
a reference point coordinator, the reference point coordinator capable of storing a multiplicity of reference points that are correlated to the multiplicity of instruments;
a reference point overlayer, that the reference point overlayer operably coupled to a receiving display device;
such that the sequential images of the image sender are visually presented on the receiving display with the reference points overlaid on the receiving display device.

2. The apparatus in claim 1 where the image frame grabber is a camera that is incorporated within the transmitting display device.

3. The apparatus in claim 1 where the image annotator is a stylus that physically interacts with the transmitting display device.

4. The apparatus of claim 1 where the image annotator transmits a multiplicity of X, Y JSON coordinates from the transmitting display device to the receiving display device.

5. The apparatus of claim 1 where the image annotator displays the annotation in the form a visual display.

6. The apparatus of claim 1 where the visual display can be modified to show any desired dimensional changes.

7. The apparatus of claim 1 in which the transmitting display device and receiver display device communicate using an internet enabled communication protocol.

8. The apparatus of claim 5 in which the internet enabled communication protocol is WebRTC.

9. A system for the storage of virtual annotations that identify features on medical devices comprising:
a means for capturing and electronically communicating through a cloud-based network series of user annotated surgical instrument feature overlays captured from a sender; and
a means for retrieving from the sender the series of user annotated surgical instrument features and displaying on a receiver captured from the sender;
so that the images that are displayed on the receiver are similar to those that are displayed on the sender with the user annotated surgical instrument feature overlays.

10. The system of claim 7 whereby the cloud-based database interacts with the sender and the receiver using WebRTC.

11. The system of claim 7 whereby the cloud-based database uses X, Y JSON coordinates.

12. A method for the virtual annotation of medical devices comprising:
capturing, storing, and displaying a series of image frames on a first mobile display device by an integrated camera;
receiving user annotations on the first mobile display device of the image frames that identify and correlate features on the image frames;
reprojecting and overlaying those annotations on the first mobile display device;
storing on the first mobile display device or the second mobile display device the series of image frames and the annotations;
transmitting the series of image frames and annotations to a second mobile display device;
displaying the series of image frames and annotations on a second mobile display device;
so that a viewer of the second mobile display device observes in near real time the series of image frames that were captured by the integrated camera.

13. The method of claim 10, in which the user annotations are stored as X, Y JSON coordinates.

14. The method of claim 11, in which the second mobile display device is continuously monitoring for X, Y JSON coordinate changes.

15. The method of claim 11 in which the second mobile display device can take one or more still image snapshots of one or more image frames that were captured by the integrated camera of the first mobile display device.

16. The method of claim 11 in which the second mobile display device can take one or more still image snapshots of one or more image frames that were captured by the integrated camera of the second mobile display device.

17. The method of claim 11 in which the still image snapshots are stored and may be further annotated on the second mobile display device.

18. The method of claim 11 in which the image frames appear sequentially on the second mobile display device.

19. The method of claim 11 in which one or more still image snapshots of one or more image frames and the annotations can be saved on to an image sequence.

20. The method of claim 11 in which the image reel with the annotated images are erased after a session.
